(19) United States
(12) Patent Application Publication
(10) Pub. No.: US 2005/0101944 A1
(43) Pub. Date: May 12, 2005

(54) Laser radiofrequency hybrid ablation needle

(76) Inventor: Jason R. Williams, Orange Beach, AL (US)

Correspondence Address: Matthew Burr;Lake Austin Marina, Ste. 200, 2219 Westlake Drive, Austin, TX, 78746, US

(21) Appl. No.: 10/944,677

(22) Filed: Sep. 17, 2004

Related U.S. Application Data
(63) Continuation-in-part of application No. 06/507,649, filed on Jun. 27, 1983, now Pat. No. 4,613,963.

(51) Int. Cl. A61B 18/20; A61B 18/14

(52) U.S. Cl. 606/15; 606/41

(57) ABSTRACT

A laser radiofrequency hybrid ablation needle for the destruction of unwanted human tissue. The needle is introduced into unwanted tissue. The tissue is destroyed from the thermal energy generated from the laser and RF energy. The device includes an introducing needle. The needle contains multiple smaller needles (tines or array). The introducing needle also contains a laser filament. The introducing needle is placed into the unwanted tissue using image guidance such as computed tomography. The laser filament and needle array are advanced out of the the end of the introducing needle and into the unwanted tissue. The introducer needle extends from hollow handle. Inside the hollow handle are housed the RF tine array and the laser fibers. The handle has a button or lever to push the tines and laser fiber out of the needle end. The RF tines are thin needles that conduct the RF energy. The tine ends spread out when pushed out of the end of the needle. RF energy is thus conducted over a greater surface area of tissue. The laser fiber (such as a fiber optic needle) carries laser light energy. The laser fiber can be advanced out of the end of the needle. The fiber can be used to carry laser energy from various types of lasers.

The present disclosure claims priority from the provisional application of the same title and by the same inventor, filed Oct. 1, 2003, with provisional application Ser. No. 06/507,649.

### FIELD OF THE INVENTION

The present invention relates generally to tissue ablation needles and more specifically to a laser radiofrequency (RF) hybrid ablation needle for the destruction of unwanted human tissue. Unwanted tissues include, but are not limited to tumors. The needle system of the present invention is introduced into unwanted tissue (i.e., a solid tumor mass) Thermal energy generated from the combination laser and RF energy destroys the mass.

### BACKGROUND OF THE INVENTION

Tissue ablation needles have been in therapeutic use for years. An example of a tissue ablation needle is that available from Boston Scientific, such as model RF 3000 and the RITA RF ablation system. There are also several laser systems available. The laser systems can be used for tissue ablation when attached to a fiber needle system such as, for example, that made by FiberTech (FiberTech Group; Berlin, Germany).

A problem with conventional tissue ablation needles are that they use only one or the other of radiofrequency or laser energy for the generation of heat. Another problem with conventional tissue ablation needles are that it takes a long time to perform a tissue ablation to completion. Another problem is that conventional tissue ablation needles use grounding pads to conduct safely the energy in the radiofrequency ablation systems. If too much energy is used in an effort to speed up the ablation, a skin burn could occur from the grounding pads. Laser ablations systems do not require ground pads, but can ablate only a small amount of tissue when used alone.

While these devices are suitable for the particular purpose to which they are addressed, they are not optimized for the destruction of unwanted human tissue, including but is not limited to, cancer. The energy in the radiofrequency ablation systems, for instance, can result in burns if too much energy is used in an effort to speed up the ablation. Laser ablations systems can ablate only a small amount of tissue when used alone.

In these respects, the laser/radio-frequency hybrid ablation needle according to the present invention substantially departs from and improves the conventional concepts and designs of the prior devices, and in so doing the invention provides an apparatus primarily developed for the purpose of the efficient and safe destruction of unwanted human tissue such as cancer. The needle system of the present invention is introduced into unwanted tissue (i.e., cancerous tumor) and the tissue is destroyed from the thermal energy generated from the combination of laser and RF energy.

### BRIEF DESCRIPTION OF THE DRAWINGS

Various other objects, features and attendant advantages of the present invention will become fully appreciated as the same become better understood when considered in conjunction with the accompanying drawings, in which like reference characters designate the same or similar parts throughout the several views, and wherein:

FIG. 1 is a side view lateral cross section of the present invention.

FIG. 2 is an exploded view of the embodiment of FIG. 1.

FIG. 3 is a side view of an alternative embodiment of the invention showing a variation of the needle tine array.

FIG. 4 is a side view of an alternative embodiment of the invention with a plurality of laser filaments.

FIG. 5 a side view of an alternative of the embodiment of FIG. 4.

### DETAILED DESCRIPTION OF THE INVENTION

In view of the foregoing disadvantages inherent in the known types of tissue ablation needle now available to practitioners of the art, the present invention provides a new laser/radiofrequency hybrid ablation needle construction wherein the same can be utilized for the safe and efficient destruction of unwanted human tissue.

The general purpose of the present invention is to provide a new laser/radiofrequency hybrid ablation needle that has many of the advantages of the tissue ablation needle mentioned heretofore and many advantageous features that result in an improved laser/radiofrequency hybrid ablation needle.

To attain the advantages described herein, the present invention generally provides an introducing needle. The needle contains one or more smaller needles (tines or array). The introducing needle also contains a laser filament. The introducing needle is placed into the unwanted tissue using image guidance such as computed tomography. The laser filament and needle array are advanced out of the distal end of the introducing needle and into the unwanted tissue. The introducer needle is a hollow needle with a handle on the proximal end. Inside the hollow needle is the RF tine array and the laser fiber. The handle has a button, lever, or other functional mechanism to push the tines and laser fiber out of the distal needle end. The RF tines are small needles that conduct the RF energy. The tine ends spread out when pushed out of the end of the needle. This allows the RF energy to be conducted over a greater tissue surface area. The laser fiber carries the laser light energy (such as a fiber optic needle). The laser fiber can be advanced, for example, out of the distal end of the needle. The optic fiber carries laser energy from various types of lasers of a selected energy output.

There has thus been described, rather broadly, the general features of the invention in order that the detailed description thereof may be better understood, and so that the present contribution to the art may be better appreciated. Practitioners of the art will appreciate additional features of the invention, which are described below.

In this respect, before explaining at least one embodiment of the invention in detail, it is to be understood that the invention is not limited in its application to the details of construction and to the arrangements of the components set forth in the following description and illustrated in the drawings. The invention is capable of other embodiments and of being practiced and carried out in various ways. Also, it is to be understood that the phraseology and terminology employed herein are for the purpose of the description and should not be regarded as limiting.

Accordingly, an object of the present invention is to provide a laser/radiofrequency hybrid ablation needle that overcomes the shortcomings of the prior devices.

Another object of the present invention is to provide a laser/radiofrequency hybrid ablation needle for the destruction of unwanted human tissue, including but not limited to, cancer. The needle system is introduced into unwanted tissue (i.e. cancerous tumor). This tissue is destroyed from the thermal energy generated from the combined laser and RF energy.

Yet another object is to provide a laser/radiofrequency hybrid ablation needle that utilizes radiofrequency energy along with laser light energy to generate tissue destruction.

Still another object is to provide a laser/radiofrequency hybrid ablation needle that generates tissue destruction faster than radiofrequency or laser energy alone.

A further object is to provide a laser/radiofrequency hybrid ablation needle that with the ability to ablate a larger area of tissue than either radiofrequency (RF) systems or laser systems alone.

A still further object is to provide a laser/radiofrequency hybrid ablation needle that allow for complete tissue destruction.

Other objects and advantages of the present invention will be appreciated by those skilled in the art, and it is intended that such other objects and advantages are within the scope of the present invention.

To accomplish the above and related objects, the invention may be embodied in the forma illustrated in the accompanying drawings, attention being called to the fact, however, that the drawings are illustrative only, and that changes within the scope of the invention may be made in the specific construction illustrated.

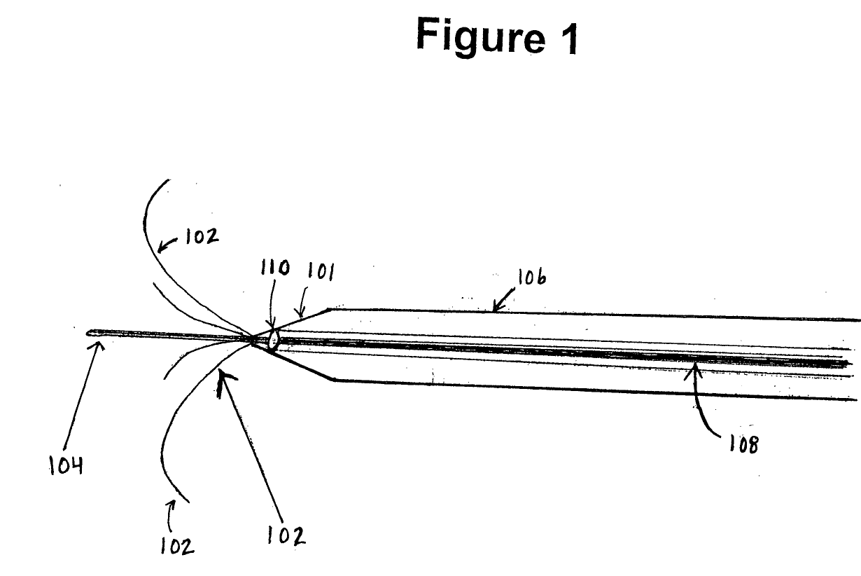

Turning now to the drawings, FIG. 1 is a side view lateral cross section of the present invention. A laser/radiofrequency hybrid ablation needle of the present invention is depicted. Introducing needle 101 contains multiple smaller needles (tines or array) 102. Introducing needle 101 also contains laser filament 104. Introducing needle 101 is placed into the unwanted tissue using image guidance such as computed tomography. Laser filament 104 and needle array 102 are advanced out of the distal end of introducing needle 101 and into the unwanted tissue (not shown). Distal introducer needle 101 is contiguous with hollow handle 106 on the proximal end. Inside hollow handle 106 is RF tine 102 array and the laser fiber 104. Handle 106 has a button or lever (not shown) to push tines 102 and laser fiber 104 out of distal needle end 101. RF tines 102 are thin, usually flexible, needles that conduct the RF energy. The distal ends of the tines spread out when pushed out of the end of the needle. The spreading allows the RF energy to be conducted over a greater surface area of tissue. Laser fiber 104 (such as a fiber optic needle) carries laser light energy to the tissue. Laser fiber 104 can be advanced out of the distal end of needle 101. The optic fiber carries laser energy from one or more of a selected type of laser, depending on the coherence and/or energy desired by the practitioner or necessitated by the tissue to be ablated.

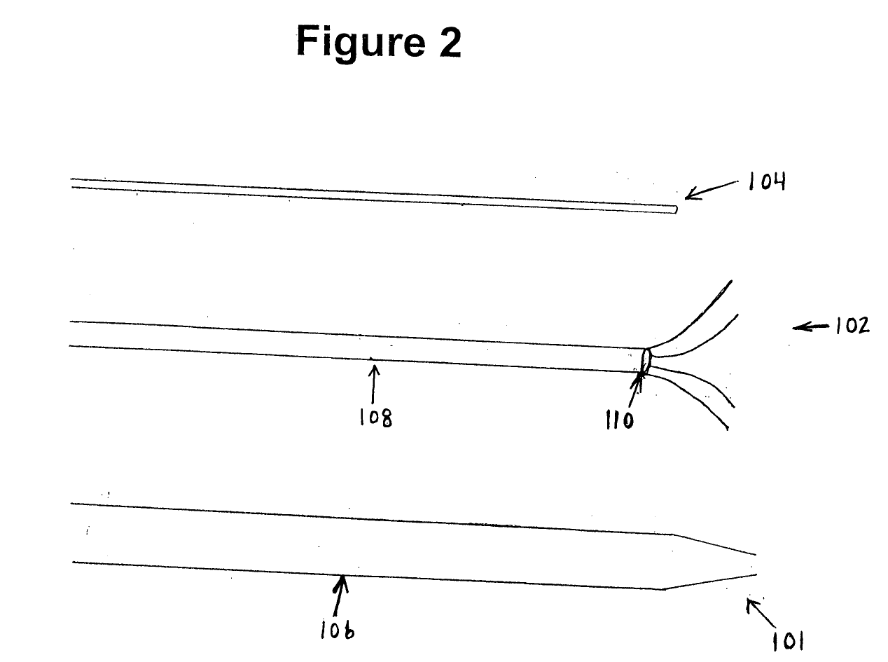

FIG. 2 is an exploded view of the embodiment of FIG. 1. Introducer needle 101 is contiguous with hollow handle 106. Housed inside hollow handle 106 is RF tine 102 array and laser optic fiber 104. Handle 106 has a button or lever (not shown) which is used to push tines 102 and laser fiber out 104 of the needle end 101. Introducer needle 101 is punctured through the skin and advanced into the area of unwanted tissue. RF and laser energy are then applied in combination to the tissue. The RF energy is conducted through the tines, which generate heat in the tissue. The laser energy is conducted through the optical filament, also generating heat within the tissue. Introducer needle 101 can be of varying sizes. It can be made from many types of metals and plastics, Needle 101 can have various curves and bends. Needle 101 has one or more filament exit hole 110 at any desired location. Introducer needle 101 can have various types of handles 106. Handle 106 provides a system (not shown) for advancing and retracting laser filament 104 and RF tine 102 array out of end 101.

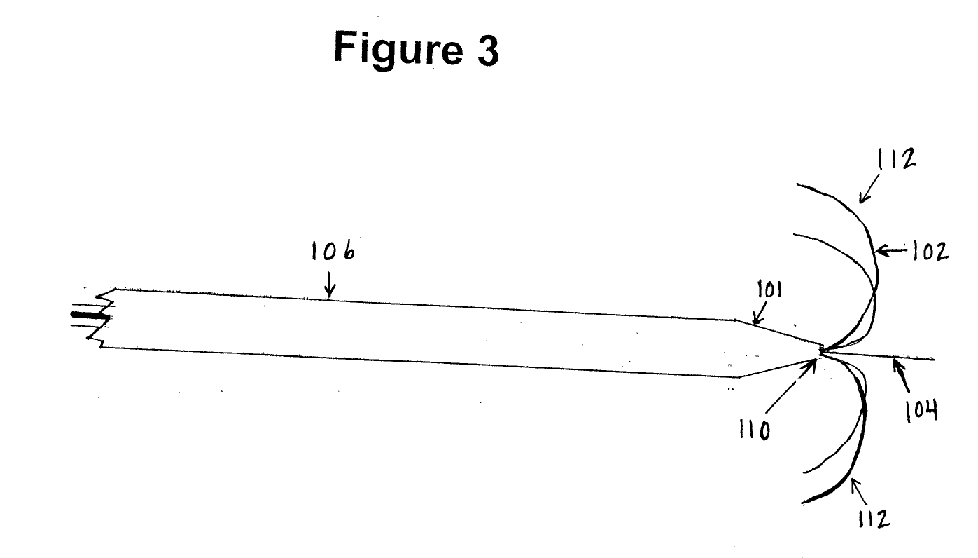

FIG. 3 is a side view of an alternative embodiment of the invention showing a needle tine array 112. RF tines are thin needles that conduct the RF energy. Tines 102 spread out when pushed out of end 110 of needle 101. This allows the RF energy to be conducted over a greater surface area. RF tines 102 are thin needles that can be advanced out end 110 of introducer needle 101. The multiple tines make array 112. Array 112 increases the dispersion of the RF energy. Tines 102 can be single or multiple (e.g., up to ten or more) in number. Tines 102 can be straight or have varying degrees of curvature. Tines 102 can be made from various types of metal or other thermal conductor.

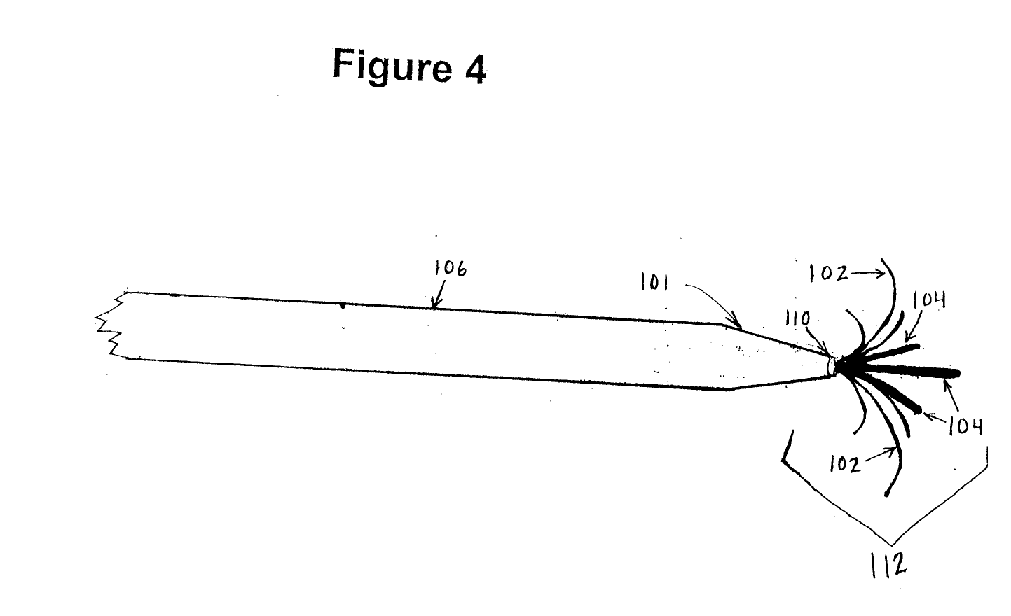

FIG. 4 is a side view of an alternative embodiment of the invention with a plurality of laser filaments. Laser fibers (such as a fiber optic needle) 104 carry laser light energy. Laser fibers 104 can be advanced out of end 110 of needle 101. Optic fiber 104 can carry laser energy from various types of lasers. Laser fiber 104 is a fiber optic filament. The filament conducts the laser energy. The filament allows for the conduction of laser energy out of end 110 of needle 101. The filament is advanced into tissue. A laser beam is conducted through the filament and deposited the laser energy (heat) into the surrounding tissue. The laser energy generates heat that destroys the tissue adjacent to the filament. There can be single or multiple laser filaments which can be advanced out of the introducing needle tip. The filament can be constructed of various materials which allow conduction of laser energy. Various types of laser energy can be used. The filament can be of various sizes aid lengths.

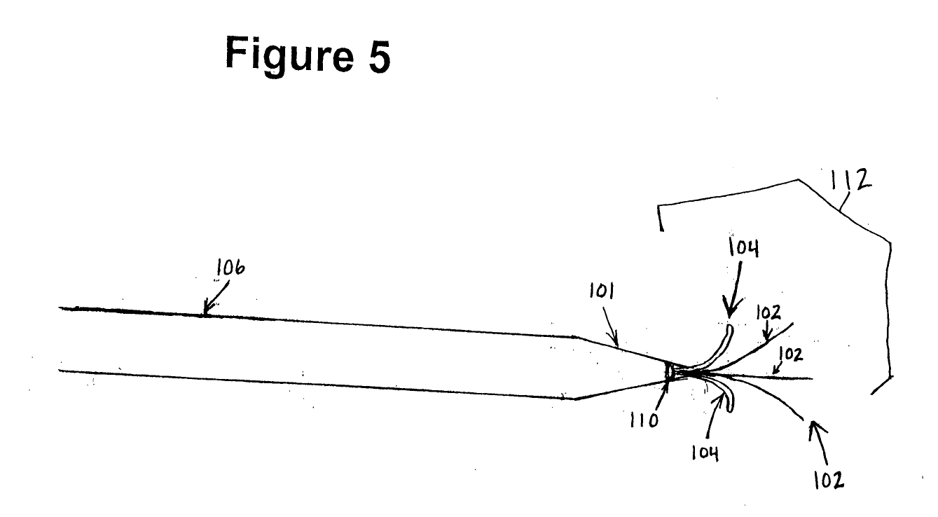

FIG. 5 a side view of an alternative of the embodiment of FIG. 4. handle 106 is hollow and houses inside RF tine array 112 and laser filaments 104. A system in the handle of the introducer needle (push button or slide bar, etc) advances RF tine array 112 and laser filaments 104 out of end 110 of needle 101. Introducer needle 101 is advanced into the area of tissue that is to be ablated. RF tine array 112 and laser filaments 104 are advanced out of end 110 of introducer needle 101 into the tissue. RF and laser energy are applied in combination, or serially, to the tissue. RF tines 102 generate heat from conducting RF energy. Laser filaments 1041 generate heat from laser energy. The heat generated from these two energy sources and modalities results in the destruction of the tissue adjacent to RF tine array 112 and laser filaments 104. RF tines 102 can be of various shapes, sizes, numbers, and lengths so as to allow for different sizes and configuration of RF tine array 112 when deployed from introducer needle tip 110. laser filaments 104 can be of various shapes, sizes, numbers, and lengths. Introducer needle 101 can be various sizes and lengths. All components can be made from many different materials of suitable optical, thermal and biocompatible properties.

The laser/RF hybrid needle system herein described allows for the destruction of unwanted tissue. The needle is introduced into the unwanted tissue (usually cancer). The RF tines and laser filament are advanced out of the needle tip and into the unwanted tissue. When RF energy is applied to the tines it causes heating of the tissue. The heat is generated from frictional forces in the ions adjacent to the tines. The laser filament conducts the laser energy and generates heat by laser light energy. The heat from laser and RF energy results in destruction of the adjacent tissue.

Implied in the above description is that each of the RF tines are connected to an RF energy supply, either internal or external to handle 106, and, likewise, each optical fiber is connected to an internal or external laser (electromagnetic) energy supply. With respect to the above description, it will be appreciated by those of skill in the art that the optimum dimensional relationships for the parts of the invention, to include variations in size, materials, shape, form, function and manner of operation, assembly and use, are deemed readily apparent. All equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention and the appended claims.

1. An apparatus to ablate tissue, the apparatus comprising: a housing; an introducing needle extending distally from the housing and having one or more exit holes; one or more RF tines housed within the housing and connected to an RF power supply; one or more optical fibers housed within the housing and connected to an electromagnetic power supply; and means to extend one or more of tine and one or more of optical fiber out of at least one exit hole into the tissue.

2. A method of ablating tissue, the method comprising the step of applying in combination RF energy and laser energy to the tissue.

3. The method of claim 2, wherein the RF energy and the laser energy are applied to the tissue serially.

4. The method of claim 2, wherein the RF energy and the laser energy are applied to the tissue at least approximately simultaneously.

5. The method of claim, further comprising the step of using guiding imagery to locate the tissue and to guide the ablating energy to the tissue.
